(19) United States
(12) Patent Application Publication
(10) Pub. No.: US 2007/0207508 A1
(43) Pub. Date: Sep. 6, 2007

(54) METHOD AND ASSAY KIT FOR SIMULTANEOUSLY DETECTING MULTIPLE TUMOR MARKERS WITH INTERFERENCE INDICATION

(76) Inventors: Jianer Yao, Shanghai (CN); Xuelei Zhou, Shanghai (CN); Chaoling Luo, Shanghai (CN); Liujuan Mao, Shanghai (CN)

Correspondence Address: DORSEY & WHITNEY LLP;INTELLECTUAL PROPERTY DEPARTMENT, SUITE 1500, 50 SOUTH SIXTH STREET, MINNEAPOLIS, MN, 55402-1498, US

(21) Appl. No.: 11/682,141

(22) Filed: Mar. 5, 2007

Related U.S. Application Data
(63) Continuation of application No. PCT/CN05/01409, filed on Sep. 5, 2005.

(30) Foreign Application Priority Data
Sep. 3, 2004 (CN) CN 200410054257.1

(51) Int. Cl. G01N 33/574

(52) U.S. Cl. 435/7.23; 977/902

(57) ABSTRACT

It is disclosed a method for simultaneous detection of multiple tumor markers, comprising the steps of: (a) mixing analyte samples, a first antibody solution and a second antibody solution, thereby forming a quaternary composite of “Second Antibody-Tumor Marker-First Antibody-bead”; (b) detecting the detectable signals from the distinct beads of the quaternary composites to determine the presence of each tumor marker in the analyte sample. The method of invention can simultaneously detect multiple tumor markers qualitatively or quantitatively. It is also simple, rapid and accurate. The invention also provides the corresponding assay kit.

### CROSS-REFERENCE TO RELATED APPLICATION(S)

This application is a continuation of International application number PCT/CN2005/001409, filed Sep. 5, 2005 which claims priority to Chinese application No. CN 200410054257.1 filed Sep. 3, 2004, the contents of both are herein incorporated in their entirety by reference.

### FIELD OF THE INVENTION

The present invention relates to in vitro diagnostics fields and, in particular, relates to a method and assay kit for simultaneously detecting multiple tumor markers with interference indication.

### BACKGROUND OF THE INVENTION

Tumors or cancers are great threats to human health with high death rate. It would be of great excitement to find the tumor markers to be able to screen for and diagnose cancer early. So far, the precision and accuracy of tumor markers assay results are of essential importance to patient's health.

Luminex xMAP features a flexible, open-architecture design, which can be configured to perform a wide variety of bioassays quickly, cost-effectively and accurately. First, Luminex color-codes tiny beads, called microspheres (Beads), into different distinct sets. Each bead set can be covalently coated with nuclear probes (complement chains) or proteins as antigen or antibodies, allowing the capture and detection of specific analytes from a sample. After mixing with the sample analytes as antigens, antibodies or enzymes in the serum or PCR products and the detectors labeled with phycoerythrin (PE), the distinct sets of microspheres line up in a single file to pass though the detection chamber. A first laser excites the internal dyes that identify each bead set with either nuclear or antibodies (qualitative analysis) and a second laser excites any fluorescence to determine the PE concentration captured during the assay (quantitative analysis). The intensities of the PE fluorescence associated with the binding of the analyte can be converted to digital results by computer.

In the prior art, tumor marker assay for single or multiple detection is basically upon the principle of direct sandwich capture assay which is so familiar to artisan in this art. While the sandwich method always involves a wash step to remove free or unbound analytes as antigen or antibodies which would severely interfere with the measurement result. The severest interference factor which would even often result in false negative is the High Dose Hook Effect (HD-HOOK). In a two-site sandwich immunoassay, when the concentration of analyte begins to exceed the amount of antibody, HD-HOOK occurs and the dose response curve will flatten (plateau) and with further increase to the high dose region which may paradoxically become negatively sloped so that it looks like a hook in a phenomenon termed “High Dose Hook Effect” (Miles LEM, Lipschitz D A, Bieber C P and Cook J D: Measurement of serum ferritin by a 2-site immunoradiometric assay. Analyt Biochem 61:209-224, 1974.). The molecular mechanism of HD-HOOK includes the hypothesis of “Molecule Isometrics” and “The Effect of Concentrations” etc.

High Dose Hook Effect often occurs in immunoassays, covering 30% of the total positive samples. The difficult determination of sample result whether it is a high dose or a low concentration sample would often result in misdiagnosis, especially the high growing rate of false negative.

As mentioned above, the immunoassays for tumor markers in the art always involve a wash step, but the interference caused by Heterophile Antibodies would neither be removed nor indicated only by washing. The interference of Heterophile Antibodies that cause false-positive results in two-site immunoassays is by bridging the capture and detection antibodies and thus blocking the reactive site from binding the analyte of interest. The common Heterophile Antibodies include HAMA (Human anti-mouse Antibody) antibodies and rheumatoid factors etc.

HAMA (Human anti-mouse Antibody) is a well-recognized cause of heterophile antibodies interference in immunoassays with an occurring rate of 10%. E.g, human or patients receiving monoclonal antibodies treatment would develop HAMA antibodies in their serum. Since the serum sample from these subjects contains human anti-mouse antibody, it is called as “HAMA SERUM”. In the sandwich immunoassays, when the applied capture and detection antibodies are both monoclonal, the HAMA antibodies in the “HAMA SERUM” would bridge them together thus blocking the reactive site from binding the analyte of interest, thereby producing a false-positive signal (Hazra D K, Britton K E, Lahiri V L, Gupta A K, Khanna P, Saran S. Nucl Med Commun. 1995 February; 16(2): 66-75.). While the common immunoassays in the art can not discriminate HAMA SERUM from normal serum, it is difficult to determine whether the measurement is true positive or false positive so that it is easy to result in misdiagnosis.

The other common interference cause by heterophile antibodies is Rheumatoid Factors (RF). The cause is the same as HAMA interference.

Summing up, the conventional immunoassay methods always involve disadvantages such as complex operation, time consuming, especially in the single measurement of tumor marker; as well as indiscrimination of true positive from false positive. Thus, there is an urgent need in the art to develop a new method and assay kit for simultaneous detecting multiple tumor markers in simplified operation (no washing step), especially with HD-HOOK and heterophile antibodies interference indication.

### SUMMARY OF THE INVENTION

Accordingly, one object of the present invention is to provide a new method and assay kit for simultaneous detecting multiple tumor markers, which is easily operated (no washing step) and has high sensitivity, good specificity and well precision, so that one can combine reaction with measurement results output in one complete operation. Moreover, this method can indicate the HD-HOOK and heterophile antibodies interference.

In the first aspect of the invention, it is provided a new method for simultaneously detecting multiple antigens, which are tumor markers, wherein the method comprises the following steps:

(a) mixing an analyte sample with a first antibody solution and a second antibody solution to form reaction mixture (a),

wherein mixing the sample with the first and the second antibody solutions can be conducted in order or simultaneously;

the first antibody solution contains 2 to 50 kinds of different antibodies, each first antibody is an antibody specific to tumor markers respectively and is coupled with distinct microsphere to form the composite as shown in Formula (I),
Anti₁X-bead (I)

wherein X represents a tumor marker, “Anti₁X” represents the first antibody that specific to the tumor marker X, and “bead” represents a microsphere and “-” represents a covalent bond;

the second antibody solution contains 2 to 50 kinds of different antibodies, each second antibody is specific to a tumor marker and corresponds to a first antibody in the first antibody solution, and each pair of the first antibody and second antibody can simultaneously bond to the corresponding tumor marker; and the mole ratio of first antibodies and the second antibodies is from 1:0.1 to 1:2;

thereby forming a quaternary composite of “Second antibody-Tumor Marker-First antibody-bead” in the reaction mixture (a);

(b) detecting the detectable signals from the bead of the quaternary composite to determine the presence of each tumor marker in the sample.

In a preferred embodiment, it further comprises the step (c): comparing the detectable signal measured with the control or the calibration curve to determine the presence and/or the amount of the tumor markers in the analyte sample.

In another preferred embodiment, the detectable signal is fluorescence.

In another preferred embodiment, the bead is a color coded polystyrene microsphere with a diameter of 2 to 10 μm.

In another preferred embodiment, the first antibody solution and the second antibody solution respectively contain the grouped first antibodies and the grouped second antibodies specific to the grouped tumor markers selected from the group consisting of:

(i) Alpha-Fetoprotein (AFP), Carcinoembryonic antigen (CEA), Carcinoma antigen 125 (CA125), Carbohydrate antigen 199 (CA199), total Human prostate antigen (PSA), free Human prostate antigen (f-PSA), Neuron specific enolase (NSE), Carbohydrate antigen 242 (CA242), Cancer antigen 153 (CA153), beta human Chorionic Gonadotropin (β-HCG);

(ii) Alpha-Fetoprotein (AFP), Carcinoembryonic antigen (CEA), Carcinoma antigen 125 (CA125), Carbohydrate antigen 199 (CA199), Carbohydrate antigen 72-4 (CA72-4), Carbohydrate antigen 50 (CA50);

(iii) Carcinoembryonic antigen (CEA), Carcinoma antigen 125 (CA125), Neuron specific enolase (NSE), beta human Chorionic Gonadotropin (β-HCG), Carbohydrate antigen 50 (CA50), Squamous cell carcinoma antigen (SCCA), Cytokeratin 19 (CYFRA21-1);

(iv) Carcinoembryonic antigen (CEA), Carcinoma antigen 125 (CA125), Cancer antigen 153 (CA153), beta human Chorionic Gonadotropin (β-HCG), Squamous cell carcinoma antigen (SCCA); and

(v) total Human prostate antigen (PSA), free Human prostate antigen (f-PSA).

In another preferred embodiment, the concentration of each first antibody is from 1 to 100 ug/ml in the first antibody solution; and the concentration of each second antibody is from 0.1 to 200 ug/ml in the second antibody solution.

In another preferred embodiment, the step (a) further includes the adding HD-HOOK indication bead into the reaction mixture (a), wherein the HD-HOOK indication bead comprises a binary composite of “target antigen-beads” as shown in Formula (II),
X-bead′ (II)

wherein the “bead′” represents a microsphere different from the above “bead” in Formula (I), and “-” represents the linkage between X and “bead′”;

thereby forming a ternary composite of “Second antibody-Antigen-Indication bead′” under the existence of the second antibody having detectable signals;

Further, step (b) comprises detecting the signal on the bead of the quaternary composite and comparing the signals with the standard value or the calibration curve to determine the presence and/or the amount of the target antigen in the analyte sample.

Further, detecting the signal on the bead of the ternary composite and comparing the signals of HD-HOOK indication bead with the normal value with no HOOK effect, wherein if the signal of the HOOK bead is smaller than the normal signal, it is indicated that the measurement result is incredible; and if the signal of the HOOK bead is larger than or equivalent to the normal value, it is indicated that the concentration of the target antigen is in the measurable range.

In another preferred embodiment, in step (c), if the measured signal of the indication bead [symbol] 0.9 fold of normal value of the indication bead, then it's a high dose sample with HOOK effect.

In another preferred embodiment, the applied mole ratio of the first antibodies and the second antibodies is from 1:0.1 to 1:2.

In another preferred embodiment, the normal value of the indication bead is determined by using the method comprising the following steps:

(a′) mixing the standard solutions which contain the targeted antigen having known concentrations within the range of the measurement curve, beads solution and the second antibody solution containing the second antibodies having detectable signals, thereby forming a reaction system or mixture in which a quaternary composite of “Second antibody-antigen-First antibody-bead” is formed;

(b′) adding the HD-HOOK indication bead to the reaction mixture of step (a′), thereby under the existence of the second antibodies with detectable signals, forming a ternary composite of “second antibody-antigen-bead′” for different concentration of target antigen in the standard solution;

(c′) measuring the detectable signals on the bead of the ternary composite and defining P+2SD as the normal value, wherein “P” represents the average value of the bead signals in the ternary composite for the different concentrations of target antigen standard; and “2SD” represents 2 times of the standard deviations of the beads signals.

In another preferred embodiment, the step (a) further includes adding heterophile antibody Interference indication bead to the reaction mixture (a), wherein the heterophile antibody Interference indication bead is a binary composite of “heterophile antibodies Interference Catcher-bead” as shown in Formula (III),
Z-bead″ (III)

wherein “Z” represents the heterophile antibodies Interference Catcher, “bead″” represents a microsphere different from the above “bead” and “bead′”, and “-” represents the linkage between “Z” and “bead″”,

thereby forming a quaternary composite of “Second antibody—Heterophile Antibodies—Heterophile Antibodies Catcher-bead″” when the Heterophile Antibodies exist in the sample;

Further, step (c) comprises: detecting the detectable signal on the bead of the quaternary composite and comparing the signals with the standard value or the calibration curve, thereby determining the presence and/or the amount of the target antigen in the analyte sample.

Moreover, detecting the detectable signal on the bead of the quaternary composite of “Second antibody—Heterophile Antibodies—Heterophile Antibodies Catcher-bead″” and comparing the signals of Heterophile Antibodies Catcher-bead″ with the normal value (without Heterophile Antibodies interference effect), wherein if the signal of the Heterophile Antibodies Catcher-bead″ is >1.5 times of the normal value, it is indicated that the measurement result is incredible; and if the signal of the Heterophile Antibodies Catcher-bead″ is [symbol] 1.5 times of the normal value, it is indicated that there is no interference of the Heterophile Antibodies in the sample.

In another preferred embodiment, if the signal of the Heterophile Antibodies Catcher-bead″ is >2 times of the normal value, then the measurement result of the target antigen is incredible, wherein the normal value is determined by using the method comprising the following steps:

(a′) mixing the standards solutions which contains the targeted antigen having known concentrations within the range of the measurement curve, beads solution and the second antibody solution containing the second antibodies having detectable signals, thereby forming a reaction system or mixture (a′) in which a quaternary composite of “Second antibody-antigen-First antibody-bead”;

(b′) adding the Heterophile Antibodies Catcher-bead to the reaction mixture of Step (a′);

(c′) measuring the detectable signals of the Heterophile Antibodies Catcher-bead″ and defining P+2SD as its normal value, wherein “P” represents the average value of the Heterophile Antibodies Catcher-bead signals for the different concentration of target antigen standard; and “2SD ” represents 2 times of the standard deviations of the beads signals.

In another preferred embodiment, the kinds of the target antigens are 1-1000.

In another preferred embodiment, the “bead”, “bead′” and “bead″” are microspheres having different fluorescence.

In another preferred embodiment, the linkage between beads and the first antibody, Heterophile Antibodies Catcher or antigens comprises covalent bond, ligand reaction or non-specific affinity.

In another preferred embodiment, in step (b), the detection is carried out by using Luminex xMAP method.

In the second aspect of the invention, it is provided a kit for detecting multiple tumor markers, which comprises the following components:

(a′) a first vessel which contains a first antibody solution, wherein the first antibody solution contains 2 to 50 kinds of different first antibodies, and each first antibody is specific to a tumor marker respectively and is coupled onto distinct set of microsphere to become a binary composite of first Antibody-bead as shown in Formula (I),
Anti₁-bead (I)

wherein X represents a tumor marker, “Anti₁X” represents the first antibody specific to tumor marker X, “bead” represents a microsphere and “-” represents a covalent bond.

(b′) a second vessel which contains a second antibody solution, wherein the second antibody solution contains 2 to 50 kinds of different antibodies having different detectable signals, each second antibody is specific to a tumor marker and corresponds to a first antibody in the first antibody solution, each pair of the first antibody and second antibody can simultaneously bond to the corresponding tumor marker, and the mole ratio of first antibodies and the second antibodies is from 1:0.1 to 1:2.

In another preferred embodiment, the kit further includes the indication beads selected from the group consisting of:

(c′) a HD-HOOK indication bead, wherein the HD-HOOK indication bead comprises a binary composite of “target antigen-beads” as shown in Formula (II),
X-bead′ (II)

wherein the “bead′” represents a microsphere different from the above “bead” in Formula (I), and “-” represents the linkage between X and “bead′”;

(d′) a Heterophile Antibody Interference indication bead, wherein the Heterophile Antibody Interference indication bead is a binary composite of “Heterophile Antibodies Interference Catcher-bead” as shown in Formula (III),
Z-bead″ (III)

Wherein, “Z” represents the Heterophile Antibodies Interference Catcher, “bead″” represents a microsphere different from the above “bead” and “bead′”, and “-” represents the linkage between “Z” and “bead″”. The catcher includes rheumatoid factors or antibodies from mouse, chick, rabbit, goat, house, or cow.

In another preferred embodiment, the kit further includes (f′) Standards or Controls for quality control.

In the third aspect of the invention, it is provided a first antibody solution which contains 2 to 50 kinds of different first antibodies, each first antibody is specific to a tumor marker respectively and is coupled with distinct microsphere to form the binary composite of first antibody as shown in Formula (I),
Anti₁X-bead (I)

Wherein, X represents a tumor marker, “Anti₁X” represents the first antibody that specific to the tumor marker X, and “bead” represents a microsphere and “-” represents a covalent bond;

In the fourth aspect of the invention, it is provided a use of the kit or the first antibody solution of the invention, which is used to in an in vitro detection of the presence of tumor markers in a sample.

### DETAILED DESCRIPTION

After extensive and intensive research, the inventors of this present invention have found that by the adjusting of the concentration of the first antibody and the second antibody, we can get precise measurement results simultaneously of multiple tumor markers though a simplified, rapid and effective method without a washing step. Further, by adding HD-HOOK indication bead, the false negative caused by the high dose hook effect in two-site sandwich immunoassay can be easily eliminated, thereby improving the precision of the measurement results. Moreover, by the adding the Heterophile Antibody Interference indication bead, the false positive caused by Heterophile Antibodies Interference in two-site sandwich immunoassay can be easily eliminated, thereby improving the precision of the measurement results of two-site sandwich immunoassay.

As used herein, the terms “a first antibodies” and “the first antibody” are exchangeable, referring to an antibody that can specifically bind to a tumor maker.

As used herein, the terms “a second antibodies” and “a second antibody” are exchangeable, referring to another antibody that can specifically bind to the tumor maker. For a same tumor marker, the corresponding first antibody and second antibody are different and both can bind onto different epitopes of the tumor marker.

As used herein, the term “tumor marker” refers to a substance produced by cancer cells or sometimes normal cells in response to tumors during their occurrence and proliferation. They indicate the presence and growth of tumors. The common tumor markers include, but is not limited to: Alpha-Fetoprotein (AFP), Carcinoembryonic antigen (CEA), Carcinoma antigen 125 (CA125), Carbohydrate antigen 199 (CA199), total Human prostate antigen (PSA), free Human prostate antigen (f-PSA), Neuron specific enolase (NSE), Carbohydrate antigen 242 (CA242), Cancer antigen 153 (CA153), beta human Chorionic Gonadotropin (β-HCG).

Basic Principles

(a) Principle of Antibodies Sandwich Immunoassay

The principle of antibodies sandwich immunoassay is familiar to the artisans in this art. In a conventional technique, a first antibody is absorbed onto a solid surface, then the antigen binds to the first antibody. After a washing step, the antigen is reacted with a second antibody linked with enzyme is added. After another washing step, a chemical fluorescence reaction or an enzyme linkage reaction is conducted so as to detect the signals.

(b) Principles of Luminex xMAP method

As a basis for this invention, Luminex xMAP features a flexible, open-architecture design, which can be configured to perform a wide variety of bioassays quickly, cost-effectively and accurately. First, Luminex color-codes tiny beads, called microspheres (Beads), into several distinct sets. Each bead set can be covalently coated with nuclear probes (complement chains) or proteins as antigen or antibodies specific to a particular bioassay, allowing the capture and detection of specific analytes from a sample. After mixing with the sample analytes as antigens antibodies or enzymes in the serum or PCR products and the detectors labeled with phycoerythrin (PE), the distinct sets of microspheres line up in a single file to pass though the detection chamber, a first laser excites the internal dyes that identify each bead set with either nuclear or antibodies (qualitative analysis), a second laser excites any fluorescence to determine the PE concentration captured during the assay (quantitative analysis). The intensities of the PE fluorescence associated with the binding of analyte can be directly converted into digital results by computer.

In a preferred embodiment, the features of the distinct flow beads are fully utilized, while the concentration of the second antibody is optimized. One may mix the beads coupled with first antibody solution, serum sample or antigen standards or controls, second antibody coupled with PE solution into a reaction vessel in order or simultaneously, so that the following reactions happen:

(1) The first antibody on the bead is bound to the corresponding antigen (tumor marker) in the serum sample or standards or controls, thereby forming a ternary composite of “tumor marker-first antibody-bead”.

(2) The second antibody is bound to the corresponding antigen (tumor marker) in the serum sample or standards or controls and finally form a quaternary composite of “second antibody-tumor marker-first antibody-bead”, which includes first antibody coupled with bead-antigen in the serum sample-second antibody conjugated with PE, or first antibody coupled with bead-antigen in standards or controls-second antibody conjugated with PE.

No washing step is involved in the reaction and the fluorescence of the composite can be measured by Luminex xMAP in the liquid phase. Thus, a one-step method is established from the reaction to the output of qualitative or quantitative assay. On the Luminex xMAP Analyzer, the distinct sets of microspheres line up in a single file to pass though the detection chamber, a first or red laser excites the internal dyes that identify each bead set with antibodies (qualitative analysis), a second or green laser excites any fluorescence to determine the PE concentration captured during the assay (quantitative analysis). The intensities of the PE fluorescence associated with the binding of the analyte can be directly converted into digital results by computer.

Please refer to product instructions or references hereafter for detailed information about Luminex xMAP: (1) Cancer Chemotherapy and Pharmacology, 51: 321-327, (2) Journal of Immunological Methods, 227: 41-52; and (3) www.luminexcorp.com.

(c) The indication principle of Heterophile Antibodies Interference

In one aspect of the present invention, an antibody specific to a certain antigen is first covalently coupled with one color-coded micro bead named Bead NO.1. Then a serum sample or standards, and a second solution containing a PE-conjugated Second antibody specific to the antigen are added into the bead solution (It is the same as the method for normally measuring the concentration of the antigen without HD-HOOK indication). the following reactions happen:

(1) The first antibody on the Bead NO.1 is bound onto the antigen in the serum sample or standards.

(2) The second antibody is also bound onto the antigen in the serum sample or standards.

Finally, a quaternary composite of “Bead NO.1 coupled with first antibody-antigen in the serum sample-second antibody conjugated with PE” or “Bead NO.1 coupled with First Antibody-antigen in standards or controls-second antibody conjugated with PE” is formed.

On the Luminex xMAP Analyzer, the distinct sets of microspheres line up in a single file to pass though the detection chamber, a first laser excites the internal dyes that identify each bead set with either nuclear or antibodies (qualitative), a second laser excites fluorescence to determine the PE concentration captured during the assay (quantitative). The intensities of the PE fluorescence associated with the binding of the analyte can be directly converted into digital results by computer.

In another aspect of the present invention, for example, for HAMA interference indication, a mouse antibody specific to HAMA is covalently coupled with a distinct color-coded microspheres named Bead NO.2. Bead NO.2 is also added into the reaction mixture at the same time with Bead NO.1. The following reactions happen:

(1) The mouse antibody on the Bead NO.2 is bound to the HAMA antibody in the serum sample.

(2) Any of the second antibodies derived from mouse is also bound to the HAMA antibodies in the serum sample.

Finally, a quaternary composite of “Bead NO.2 coupled with Mouse Antibody-HAMA Antibody-Second Antibody conjugated with PE” is formed.

On the Luminex xMAP Analyzer, both Bead NO.2 and Bead NO.1 will line up in a single file to pass though the detection chamber at the same time, a first laser excites the internal dyes that identify the bead set with Mouse Antibody; a second laser excites fluorescence to measure the intensities of the PE fluorescence to determine whether it is a HAMA sample or not.

(d) The indication principle of HD-HOOK

In another preferred embodiment, the method of the present invention can also indicate HD-HOOK effect at the same time.

Firstly, a purified target antigen is covalently coupled with a distinct color-coded bead named Bead NO.3. After the incubation of Bead NO.1 with serum sample and the PE-conjugated Second antibody, Bead NO.3 (HD-HOOK indication bead) is added into the reaction system or reaction mixture. At this time, if the concentration of the target antigen in the sample is not in the HD-HOOK region (The amount of target antigen is not too much excess to the second antibody, and party or most of the second antibodies are free from conjugation), then the following reaction happen: The antigen on Bead NO.3 is bound to the free PE-conjugated second antibody, thereby finally forming a ternary composite of “Bead NO.3 coupled with target antigen—PE-conjugated second antibody”. The ternary composite can be detected in later detection.

On the contrary, if the concentration of the target antigen in the sample is higher than the dose response curve and in the HD-HOOK region (The amount of target antigen exceeds the amount of antibody so that most of the second antibodies is bound to the target antigens, and there are no or few second antibodies free in the reaction system), it is difficult for the antigens on Bead NO.3 to meet with the free second antibody to form the ternary composite of “Bead NO.3 coupled with target antigen—PE-conjugated second antibody”. Thus the ternary composite is unable to be detected or is detected with low fluorescence intensities.

On the Luminex xMAP Analyzer, both Bead NO.3 and Bead NO.1 will line up in a single file to pass though the detection chamber at the same time, a first laser excites the internal dyes that identify the bead set with HD-HOOK indication; a second laser excites fluorescence to measure the intensities of the PE fluorescence to determine whether it is a HD-HOOK sample or not. If the intensities of the PE fluorescence on Bead NO.3 is low (e.g., <90%, preferably <50%, more preferably <30%, best preferably <10% of the normal fluorescence intensities (normal value) when the sample is of no HD-HOOK effect), then the concentration of the target antigen is in high dose region and the sample is determined to be a HD-HOOK sample.

For example, taking the tumor marker being as the target antigen, the detailed operation of HD-HOOK indication is of the same as HAMA indication except that the preparation HD-HOOK indication bead is different and the addition of the HD-HOOK indication bead after the incubation of the above reaction.

Binary composite of Fist Antibody-Bead

The binary composite of first antibody-bead forms is as shown in Formula (I),
Anti₁X-bead (I)

Wherein, X represents a tumor marker, “Anti₁X” represents a first antibody that specific to the tumor marker X, “bead” represents a microsphere, and “-” represents a covalent bond between the first antibody and the bead.

The Coupling of Fist Antibody with Bead

To obtain different kinds of Anti₁X-Beads composite, the detailed coupling protocol of the first antibodies specific to different tumor markers with beads can refer to product instructions of Luminex Corporation or its website of www.luminexcorp.com.

Various kinds of composites Anti₁X-Beads are taken and mixed in a certain amount, thereby obtaining the First antibody solution (referred to as Solution A).

Binary composite of Antigen with Bead (HD-HOOK indication bead)

By using the similar protocols mentioned above, the purified target antigen is coupled with another bead to gain binary composite of antigen with beads (that is HD-HOOK Indication bead) which is referred to as Solution H (HD-HOOK Indication Bead Suspension).

Binary composite of Mouse Antibody with Bead (HAMA Indication Bead)

By using the similar protocols mentioned above, the mouse antibody is coupled with to another bead to gain binary composite of mouse antibody-beads (HAMA Indication Bead) which is mixed with Anti1X-Beads and also referred as Solution A.

Labeling the Second Antibody

Although a number of techniques known in the art can be used to label the second antibodies, it is preferred to use fluorescence reagent as PE to conjugate through Biotin-streptavidin linkage.

In a preferred embodiment, the protocol of biotin labeling to the second antibodies comprises: purifying the second antibodies specific to different tumor markers (anti₂X, X stands for a certain tumor marker) through dialysis, adding biotin solution dissolved in DMSO solvent to the antibodies solution, incubating for a certain time against light, Dialyze removing the unreacted biotin from the antibody solution and storing the biotinylated antibodies for use.

The biotinylated anti₂Xs specific to different tumor markers are taken and mixed in a certain amount. Thereafter, streptavidin linked with PE is added, allowing the binding of Biotin to Streptavidin so as to form the fluorescence conjugated second antibodies (as PE-anti₂X) solution (referred to as Solution C).

Controls and Standards

To gain reliable measurement results, it is recommended using controls through assay procedure. It is also recommended using several standards of different concentrations containing multiple tumor markers to gain quantitative results through assay procedure.

Standards (STDₙ, n=0−5) and Controls (Ctr1, Ctr2) solutions preparation can refer to Table 1 hereunder:

TABLE 1
Preparation of Standards and Controls solution
containing multiple antigens

| Tumor Marker | | | | | | | | |
|---|---|---|---|---|---|---|---|---|
| (TM) | STD0 | STD1 | STD2 | STD3 | STD4 | STD5 | Ctr1 | Ctr2 |
| 1 | 0 | C₁₋₁ | C₁₋₂ | C₁₋₃ | C₁₋₄ | C₁₋₅ | C₁₋₆ | C₁₋₇ |
| 2 | 0 | C₂₋₁ | C₂₋₂ | C₂₋₃ | C₂₋₄ | C₂₋₅ | C₂₋₆ | C₂₋₇ |
| 3 | 0 | C₃₋₁ | C₃₋₂ | C₃₋₃ | C₃₋₄ | C₃₋₅ | C₃₋₆ | C₃₋₇ |
| 4 | 0 | C₄₋₁ | C₄₋₂ | C₄₋₃ | C₄₋₄ | C₄₋₅ | C₄₋₆ | C₄₋₇ |
| 5 | 0 | C₅₋₁ | C₅₋₂ | C₅₋₃ | C₅₋₄ | C₅₋₅ | C₅₋₆ | C₅₋₇ |
| 6 | 0 | C₆₋₁ | C₆₋₂ | C₆₋₃ | C₆₋₄ | C₆₋₅ | C₆₋₆ | C₆₋₇ |
| . | 0 | . | . | . | . | . | . | . |
| . | | . | . | . | . | . | . | . |
| . | | . | . | . | . | . | . | . |
| 40 | 0 | C₄₀₋₁ | C₄₀₋₂ | C₄₀₋₃ | C₄₀₋₄ | C₄₀₋₅ | C₄₀₋₆ | C₄₀₋₇ |

The first row in Table 1 stands for different tumor markers, STD0 means the concentrations of all different tumor markers in the standard solution are zero; STD₁ means the concentrations of all different tumor markers in the standard solutions are C₁₋₁, C₂₋₁, C₃₋₁ . . . C₄₀₋₁ relatively, STD1 becomes the second dose point of the calibration curve. The same should be applied to STD2, STD3, STD4, STD5 as above. Ctr1 means the concentrations of all different tumor markers in the standard solution are C₁₋₆ , C₂₋₆, C₃₋₆ . . . C₄₀₋₆ relatively, and the concentration amounts are all between STD0 and STD5 and useful as an internal control point. Ctr2 means the concentrations of all different tumor markers in the standard solution are C₁₋₇, C₂₋₇, C₃₋₇ . . . . C₄₀₋₇ relatively, and the concentration amounts are all between STD0 and STD5, becoming another internal control point. STD0-STD5 as well as Ctr1 and Ctr2 are composed to be Control Solutions (referred to as Solution B in the assay procedure).

Mix well the first antibody Solution, Control Solution and the Second Antibody Solution (that is Solution A, B and C) in order or simultaneously, incubate for complete reaction (at 37±5° C. for 5-100 mins for example), then analyze on the Luminex xMAP later to obtain multiple calibration curves. (The specific amount of calibration curves depends on the amount of kinds of tumor marker in the group of the assay).

Grouping of Tumor Markers

For the different characteristics of multiple tumor markers found in practice, it's a need to separate the tumor markers into several groups to obtain reliable measurement results. A preferred grouping is as follows:

(i) Alpha-Fetoprotein (AFP), Carcinoembryonic antigen (CEA), Carcinoma antigen 125 (CA125), Carbohydrate antigen 199 (CA199), total Human prostate antigen (PSA), free Human prostate antigen (f-PSA), Neuron specific enolase (NSE), Carbohydrate antigen 242 (CA242), Cancer antigen 153 (CA153), beta human Chorionic Gonadotropin (β-HCG);

(ii) Alpha-Fetoprotein (AFP), Carcinoembryonic antigen (CEA), Carcinoma antigen 125 (CA125), Carbohydrate antigen 199 (CA199), Carbohydrate antigen 72-4 (CA72-4), Carbohydrate antigen 50 (CA50);

(iii) Carcinoembryonic antigen (CEA), Carcinoma antigen 125 (CA125), Neuron specific enolase (NSE), beta human Chorionic Gonadotropin (β-HCG), Carbohydrate antigen 50 (CA50), Squamous cell carcinoma antigen (SCCA), Cytokeratin 19 (CYFRA21-1);

(iv) Carcinoembryonic antigen (CEA), Carcinoma antigen 125 (CA125), Cancer antigen 153 (CA153), beta human Chorionic Gonadotropin (β-HCG), Squamous cell carcinoma antigen (SCCA);

(v) total Human prostate antigen (PSA), free Human prostate antigen (f-PSA).

To a certain group of tumor markers, the first antibody Solution, the Second Antibody Solution and the Controls also contain the corresponding first antibodies, second antibodies or tumor markers.

For example, when the target tumor markers are Alpha-Fetoprotein (AFP), Carcinoembryonic antigen (CEA), Carcinoma antigen 125 (CA125), Carbohydrate antigen 199 (CA199), total Human prostate antigen (PSA), free Human prostate antigen (f-PSA), Neuron specific enolase (NSE), Carbohydrate antigen 242 (CA242), Cancer antigen 153 (CA153), and beta human Chorionic Gonadotropin (β-HCG);

the corresponding Solution A should contain anti₁ AFP-beads, anti₁ CEA-beads, anti₁CA125-beads, anti₁CA19-9-beads, anti₁ PSA-beads, anti₁f-PSA-beads, anti₁NSE-beads, anti, CA242-beads, anti₁CA15-3-beads, anti₁-HCG-beads;

the corresponding Solution B should contain Standard 0 (STD0) without any tumor markers; Standard 1-5 (STD1, STD2, STD3, STD4, STD5) with different known concentrations of 10 kinds of tumor markers here above; Control 1 (Ctr1) containing 10 kinds of tumor markers here above; Control 2 (Ctr2) also containing 10 kinds of tumor markers here above; and

the corresponding Solution C should contain: PE-anti₂ APEP, PE-anti₂ CEA, PE-anti₂ CA125, PE-anti₂ CA19-9, PE-anti₂ PSA, PE-anti₂ PE-PSA, PE-anti₂ NSE, PE-anti₂ CA242, PE-anti₂ CA15-3, PE-anti₂β-HCG.

The same components of Solution A, B and C should be applied to other groups here above of tumor markers.

The detection of samples

The present invention is intended for use with any sample containing tumor markers, as serum, urine, etc. Sample from serum is preferred.

In the detection, Solution A, Human Serum Sample and Solution C are mixed well in order or simultaneously, incubated for complete reactions (at 37±5° C. for 5-100 mins for example), then analyzed on the Luminex xMAP later to determine the existence of tumor markers according to their each threshold values or to obtain the concentrations of one certain or several certain tumor markers according to the multiple calibration curves.

The main advantage of the present invention is that qualitative or quantitative information of multiple targets can be obtained through one run of the assay which is sensitive, reliable, and precise with wide detection range and simplified operation.

The applied example main advantages of the present invention exit in hereunder:

Qualitative or quantitative in formations of multiple targets obtained through one run of the assay which is sensitive, reliable, and precise with wide detection range and simplified operation.

The invention is further illustrated by the following examples. It is appreciated that these examples are only intended to illustrate the invention, but not to limit the scope of the invention. For the experimental methods in the following examples, they are performed under routine conditions, e.g., those described by Sambrook. et al., in Molecule Clone: A Laboratory Manual, New York: Cold Spring Harbor Laboratory Press, 1989, or as instructed by the manufacturers, unless otherwise specified.

Antibodies or Antigens supplied hereunder:

| MATERIALS | LOT/CAT NO. | SUPPLIER |
|---|---|---|
| Anti-CA125 | M86306M (group A) | Biodesign Inc. |
| Anti-CA125 | M86294M (group B) | Biodesign Inc. |
| Antigen CA125 | 30AC20 | Fitzgerald Inc. |
| Anti-CA153 | M37901M (clone695) | Biodesign Inc. |
| Anti-CA153 | M37552M (clone552) | Biodesign Inc. |
| Antigen CA153 | 30AC16 | Fitzgerald Inc. |
| Anti-f-PSA | M86209M (total) | Biodesign Inc. |
| Anti-f-PSA | M86806M (Free) | Biodesign Inc. |
| Antigen PSA | A86878H Pure | Biodesign Inc. |
| Anti-T-PSA | M86506M (total) | Biodesign Inc. |
| Anti-T-PSA | M86209M (total) | Biodesign Inc. |
| Antigen PSA | A86878H Pure | Biodesign Inc. |
| Anti-CA199 | M8073022 | Fitzgerald Inc. |
| Antigen CA19-9 | 30AC14 | Fitzgerald Inc. |
| Anti-NSE | 9601 | Medix Inc. |
| Anti-NSE | 9602 | Medix Inc. |
| Antigen NSE | 30-AN10 | Fitzgerald Inc. |
| Anti-β-HCG | 5012 | Medix Inc. |
| Anti-β-HCG | 5006 | Medix Inc. |
| Antigenβ-HCG | A81455M | Biodesign Inc. |
| Anti-CA242 | 101-01 | Canag Inc. |
| Antigen CA242 | | Canag Inc. |
| Anti-AFP | G4 | Shanghai Secondary Military |
| | | Medical Univ. |
| Anti-AFP | C2 | Shanghai Secondary Military |
| | | Medical Univ. |
| Antigen AFP | | Biodesign Inc. |
| Anti-CEA | A1 | Shanghai Secondary Military |
| | | Medical Univ. |
| Anti-CEA | C9 | Shanghai Secondary Military |
| | | Medical Univ. |
| Antigen CEA | A86808H | Biodesign Inc. |

The microspheres (beads) are supplied by Luminex Corp with a diameter of 5.0 um. The surface of the beads are modified by carboxyl groups (—COOH). Others are regular reagents on the present market.

### EXAMPLE 1

Simultaneously measurement of 10 kinds of tumor markers (No HD-HOOK Indication and No Heterophile Antibodies Inference Indication)

1. Preparation
1.1 Remove the primary amines and other small proteins (through dialysis or gel filtration) in the first antibody solution and measure its concentration.
1.2 Precisely weight about 5 mg of N-hydroxysulfosuccinimide (NHS) to an eppendorf tube for later use (against moisture).
1.3 Precisely weight about 5 mg of N-ethyl-N′(3-dimethylainopropyl)-carbodiimide (EDC) to an eppendorf tube for later use (against moisture).
2. The activation of the micro beads
2.1 Resuspend the stock microsphere suspension by vortex for approximately about 20 seconds. Transfer 200 μl of the stock microsphere suspension (about 2.5×10⁶ beads) to a polypropylene tube.
2.2 Pellet the microspheres by micro centrifugation at 15000 rpm for 2 mins (set to 3 mins), remove the supernatant.
2.3 Resuspend the pelleted microspheres in 100 μl dH₂O by vortex approximately 20 seconds, pellet the microspheres by micro centrifugation at 15000 rpm for 2 mins, and remove the supernatant.
2.4 Repeat step 2.3.
2.5 Resuspend the pelleted microspheres in 80 μl 100 mM Phosphate Buffer solution (PBS), pH6.2 by vortex approximately 20 seconds.
2.6 Diluted the Sulfo-NHS to 50 mg/ml in dH₂O (at use).
2.7 Add 10 μl of the Sulfo-NHS solution above to the microspheres and mix gently by vortex.
2.8 Diluted the EDC to 50 mg/ml in dH₂O (at use).
2.9 Add 10 μl of the EDC solution above to the microspheres and mix gently by vortex.
2.10 Incubate for 20 minutes at 37° C. against light.
2.11 Pellet the microspheres by micro centrifugation at 15000 rpm for 2 mins, remove the supernatant.
2.12 Add 250 μl of the 50mmol/L 2-(N-morpholino) ethanesulfonic acid (MES) pH5.0 to the microspheres and mix gently by vortex.
2.13 Pellet the microspheres by micro centrifugation at 15000 rpm for 2 mins, remove the supernatant.
2.14 Repeat step 2.12, 2.13, immediately start next step.
3. Coupling, Blocking and Storage.
3.1 Add 20μg of the prepared first antibody to the resuspended microspheres. Mix coupling reaction by vortex approximately 20 seconds.
3.2 Incubate at 37° C. for 2 hours with gentle mixing by vortex at 15 minute intervals.
3.3 Add 1 ml PBS-TBN, mix by vortex 20 seconds. Pellet the microspheres by micro centrifugation at 15000 rpm for 2 mins, remove the supernatant. (PBS-TBN containing 10 mM Phosphate buffer pH7.4, 02% of TWEEN20, 1 mg/ml Bovine Serum Albumin and 0.05% Sodium Azide)
3.4 Resuspend the pelleted microspheres in 500 μl PBS-TBN by vortex approximately 20 seconds. Pellet the microspheres by micro centrifugation at 15000 rpm for 2 mins, remove the supernatant.
3.5 Resuspend the pelleted microspheres in 500 μl PBS-TBN by vortex approximately 20 seconds.
3.6 Store at 2-8° C. against light.
4. Biotinylation of Second Antibodies (anti₂X)

4.1 Preparation of second antibodies

| | |
|---|---|
| Containing small molecules as | Dialyze against 1xPBS pH 7.4 |
| sodium azide, amines like glycine; | |
| Containing large molecules as | Purify against protein A column |
| bovine serum albumin | or other column |
| Concentration calibrating | Measure OD280 (1OD280 equivalent |
| | to 0.7 mg/ml McAb) by |
| | spectrophotometer, and bring |
| | the concentration up to 2 mg/ml by |
| | 1xPBS, pH 7.4 (when concentrate, |
| | use desalting column from Pall Inc.). |

4.2 Biotinylating reaction
   Add 25 μl 1mg/ml NHSS-biotin DMSO solution into 25 μl the above second antibodies solution, mix gently and place on ice and incubate for 2 hours against light.

5. Preparation of multiple antigen Standards and Controls (Solution B)

| TM | STD0 | | STD1 | | STD2 | | STD3 | | STD4 | | Ctr1 | | Ctr2 | |
|---|---|---|---|---|---|---|---|---|---|---|---|---|---|---|
| β-HCG | 0 | mIU/ml | 0.5 | mIU/ml | 2 | mIU/ml | 20 | mIU/ml | 100 | mIU/ml | 2 | mIU/ml | 20 | mIU/ml |
| CA19-9 | 0 | U/ml | 5 | U/ml | 40 | U/ml | 200 | U/ml | 800 | U/ml | 40 | U/ml | 200 | U/ml |
| free-PSA | 0 | ng/ml | 0.5 | ng/ml | 2 | ng/ml | 20 | ng/ml | 100 | ng/ml | 2 | ng/ml | 20 | ng/ml |
| total-PSA | 0 | ng/ml | 0.5 | ng/ml | 2 | ng/ml | 20 | ng/ml | 100 | ng/ml | 2 | ng/ml | 20 | ng/ml |
| NSE | 0 | ng/ml | 5 | ng/ml | 20 | ng/ml | 60 | ng/ml | 120 | ng/ml | 20 | ng/ml | 60 | ng/ml |
| CA125 | 0 | U/ml | 40 | U/ml | 200 | U/ml | 400 | U/ml | 800 | U/ml | 200 | U/ml | 400 | U/ml |
| CA15-3 | 0 | U/ml | 1 | U/ml | 5 | U/ml | 30 | U/ml | 240 | U/ml | 5 | U/ml | 30 | U/ml |
| CA242 | 0 | U/ml | 10 | U/ml | 50 | U/ml | 200 | U/ml | 400 | U/ml | 50 | U/ml | 200 | U/ml |
| AFP | 0 | ng/ml | 5 | ng/ml | 20 | ng/ml | 200 | ng/ml | 500 | ng/ml | 20 | ng/ml | 200 | ng/ml |
| CEA | 0 | ng/ml | 5 | ng/ml | 50 | ng/ml | 200 | ng/ml | 800 | ng/ml | 50 | ng/ml | 200 | ng/ml |

Prepare Solution B by the above table with PBS, pH 7.4 solution.

6. Preparation of Solution A
   Mix 4×10⁵ of hereafter 10 different kinds of beads as anti₁β-HCG-Beads, anti₁PEree-PSA-Beads, anti₁total-PSA-Beads, anti₁NSE-Beads, anti₁CA15-3-Beads, anti₁CA19-9-Beads, anti₁CA125-Beads, anti₁ CA242-Beads, anti₁APEP-Beads, anti₁CEA-Beads respectively in 1×PBS, pH7.4 solution and bring the total volume up to 5 ml, store at 4° C. against light for later use.
7. Preparation of the Mixture of PE Conjugated Second Antibodies (Solution A)
   Mix the Biotinylated second antibodies specific to PEree-PSA, total-PSA, NSE, CA242, CA19-9, CA125, β-HCG, CA15-3, APEP and CEA respectively in 1×PBS, pH7.4 solution with a concentration of 5 μg/ml, add PE in it with a total concentration of 60 μg/ml, and bring the total volume up to 5 ml, store at 4° C. against light for later use.
8. Detection for tumor markers in digestive system of patient serum
8.1 Collect 10 blood samples of 2 ml/vial from patient, centrifuge at 5000 rpm for 5 mins and collect supernatant for later use.
8.2 Add separately Solution A, 50 μl; Standards (STD0, STD1, STD2, STD3, STD4, STD5),
   Controls (Ctr1, Ctr2) and Serum Samples (NO.1-9), each well 5 uμl; and Solution C, 50 μl to each well. Mix on a vortex. Incubate the plate at 37° C. for 40 mins.
8.3 After incubation, mix on vortex and read the plate on Lumine 100 analyzer.

8.4 Detection results refer to the chart below:

| | Sample | 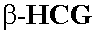 | 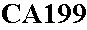 | 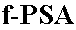 | 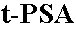 | 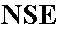 | 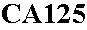 | 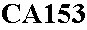 | 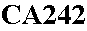 | 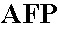 | 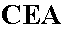 | Total |
|---|---|---|---|---|---|---|---|---|---|---|---|---|
| 1 2 3 4 5 | 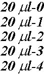 | 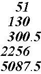 | 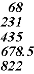 | 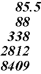 | 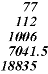 | 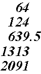 | 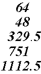 | 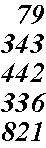 | 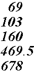 | 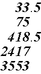 | 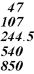 | 1286 1243 1282 1268 1359 |
| 6 | 1 | 78.5 | 196 | 104 | 81.5 | 560 | 91.5 | 123 | 75.5 | 115.5 | 102 | 1258 |
| 7 | 2 | 109 | 117 | 91 | 239.5 | 662 | 78.5 | 90.5 | 95 | 113 | 80 | 1177 |
| 8 | 3 | 71.5 | 56 | 105 | 61 | 182 | 65.5 | 86 | 68 | 82 | 97 | 1344 |
| 9 | 4 | 70 | 90 | 109 | 97 | 140 | 76 | 126 | 67 | 59 | 53 | 1229 |
| 10 | 5 | 91 | 44 | 120.5 | 401.5 | 67 | 84.5 | 116 | 49 | 35 | 60 | 1216 |
| 11 | 6 | 77.5 | 73.5 | 177 | 95 | 499 | 299 | 121 | 133 | 2187 | 99.5 | 1279 |
| 12 | 7 | 76.5 | 88 | 4397 | 10944 | 421.5 | 71 | 186.5 | 67 | 174 | 50 | 1355 |
| 13 | 8 | 39.5 | 59 | 90.5 | 56.5 | 456 | 74.5 | 136.5 | 71.5 | 60.5 | 21 | 1253 |
| 14 | 9 | 77 | 67.5 | 91.5 | 76 | 353 | 56.5 | 102 | 57 | 86 | 77 | 1250 |

NOTE: The parts in italic and bold fonts are the results of standards, others are of samples.

It's clear that the quantitative results of multiple tumor markers can be obtained simultaneously through this invention, i.e., in the chart above, there are large amount of tumor marker t-PSA in sample NO.7 which can assist clinical diagnosis.

### EXAMPLE 2

Indication of HAMA and Detection of AFP in Human Serum Samples

The mouse IgG reactive against HAMA is supplied by Biodesign Corp., the first and second antibodies specific to AFP are supplied by Shanghai Secondary Military Medical University, Antigen AFP Standard is also supplied by Biodesign Corp. and beads are supplied by Luminex Corp.
1. Preparation
1.1 Remove the primary amines and other small proteins (through dialysis or gel filtration) in the first antibody and Antigen AFP solution, measure their concentrations.
1.2 Precisely weight about 5 mg of N-hydroxysulfosuccinimide (NHS) to an eppendorf tube for later use (against moisture).
1.3 Precisely weight about 5 mg of N-ethyl-N′(3-dimethylainopropyl)-carbodiimide (EDC) to an eppendorf tube for later use (against moisture).
2 The activation of the micro beads
2.1 Resuspend the stock microsphere suspension of 33# and 46# set by vortex for approximately 20 seconds. Transfer 200 μl of the stock microsphere suspension (about 2.5×10⁶ beads) respectively to two polypropylene tubes.
2.2 Pellet the microspheres by micro centrifugation at 15000 rpm for 2 mins (set to 3 mins), remove the supernatant.
2.3 Resuspend the pelleted microspheres in 100 μl dH₂O by vortex approximately 20 seconds, pellet the microspheres by micro centrifugation at 15000 rpm for 2 mins, and remove the supernatant.
2.4 Repeat step 2.3.
2.5 Resuspend the pelleted microspheres in 80 μl 100 mM PBS, pH6.2 by vortex approximately 20 seconds.
2.6-2.14 Conduct the same as in EXAMPLE 1.
3. Coupling, Blocking and Storage.
3.1 Add 20 μg of the prepared mouse IgG to the activated 46# set beads. Add 20 μg of the prepared first antibody specific to AFP to the activated 33# set beads. Mix the coupling reactions respectively by vortex for approximately 20 seconds.
3.2 Incubate at 37° C. for 2 hours against light with gentle mixing by vortex at 15 minute intervals.
3.3 Add respectively 1 ml PBS-TBN, mix by vortex 20 seconds. Pellet the microspheres by micro centrifugation at 15000 rpm for 2 mins, remove the supernatant. (PBS-TBN containing 10 mM Phosphate buffer pH7.4, 02% of TWEEN20, 1 mg/ml Bovine Serum Albumin and 0.05% Sodium Azide) 3.4 Resuspend the pelleted microspheres in 500 μl PBS-TBN by vortex approximately 20 seconds. Pellet the microspheres by micro centrifugation at 15000 rpm for 2 mins, remove the supernatant.
3.5 Resuspend the pelleted microspheres in 500, μl PBS-TBN by vortex approximately 20 seconds
3.6 Measure the beads concentrations by microscopic examination and cell count, the results are as hereafter: 33# is 1×10⁶/ml; 46# is 1×10⁶/ml.
3.7 Store at 2-8° C. against light
4. Biotinylation of Second Antibodies

4.1 Preparation of second antibodies

| | |
|---|---|
| Containing small molecules as | Dialyze against 1xPBS pH 7.4 |
| sodium azide, amines like glycine; | |
| Containing large molecules as | Purify against protein A column |
| bovine serum albumin | or other column |
| Concentration calibrating | Measure OD280 (1OD280 equivalent |
| | to 0.7 mg/ml McAb) by |
| | spectrophotometer and bring the |
| | concentration up to 2 mg/ml by |
| | 1xPBS, pH 7.4 (when concentrate, |
| | use desalting column from Pall Inc.). |

4.2 Biotinylating reaction
   Add 25 μl 1 mg/ml NHSS-biotin DMSO solution into 10 μl the above second antibodies specific to AFP solution, mix gently and place on ice and incubate for 2 hours against light. Dialysis overnight for later use.
5. Preparation of antigen Standards (Solution B)
   Prepare AFP standards with concentrations of 0 ng/ml, 5 ng/ml, 20 ng/ml, 200 ng/ml, 500 ng/ml by PBS, pH7.4.
6. Preparation of Solution A
   Mix respectively 33# and 46# beads here above in 1 ml PBS, pH7.4 solution with concentration of 1×10⁴/ml.
7. Preparation of PE Conjugated Second Antibody (Solution C)
   Mix the Biotinylated second antibody specific to AFP in PBS, pH7.4 solution with a final concentration of 5 μg/ml, add Streptavidin linked PE with a total concentration of 60 μg/ml, and bring the total volume up to 5 ml, store at 4° C. against light for later use.
8. Indication of HAMA and Detection for AFP in Human Serum
8.1 Collect 10 blood samples, 5 with and 5 without HAMA interference of 2 ml/vial, centrifuge at 5000 rpm for 5 mins and collect supernatant for later use.
8.2 Add separately Solution B, 5 μl; 25 μl Solution A, 25 μl Solution C to each well; and add 5 μl/well of 10 human serum samples, 25 μl/well Solution A, and 25 μl/well Solution C into other 10 wells. Mix and react in incubator at 37° C. for 40 mins.
8.3 After incubation, mix on vortex and read the plate on Lumine 100 analyzer.

8.4 Detection results refer to the chart below:

Detection Results

| | Samples or known | 33# for | 46# for HAMA |
|---|---|---|---|
| | concentrations | Detection | Indication |
| Standards | 0 ng/ml | 56.7 | 90 |
| | 5 ng/ml | 250.8 | 101 |
| | 20 ng/ml | 768.8 | 110 |
| | 200 ng/ml | 4829.5 | 96 |
| | 500 ng/ml | 7439.5 | 95 |
| Samples | Sample 1 | 2845.0 | 29364.5 |
| | Sample 2 | 2875.0 | 29687 |
| | Sample 3 | 2349.0 | 25476.5 |
| | Sample 4 | 3215.0 | 32452.5 |
| | Sample 5 | 3014.5 | 31484 |
| | Sample 6 | 115.0 | 145 |
| | Sample 7 | 3478.0 | 123 |
| | Sample 8 | 257.0 | 114.5 |
| | Sample 9 | 6473.0 | 134 |
| | Sample 10 | 578.5 | 94.5 |

8.5 Determination of Results:
   The average MIF of 46# beads with standards is 113, while the MIFs of samples 1-5 are 10 times higher than which without HAMA interference. The MIFs with sample 6-10 is less than 1.5×113, which is determined to be without HAMA interference.

### EXAMPLE 3

Simultaneously measurement of 5 kinds of tumor markers (With HAMA Interference Indication)

Generally according to the steps in EXAMPLES 1&2, detecting simultaneously 5 kinds of tumor markers with HD-HOOK interference indication. Different target antigens SCCA, CA125, CA15-3,CEA, β-HCG are used. Each uses one detection beads and HAMA indication microbeads. 5 detection microbeads and 5 indication microbeads have different coding fluorescence.

Moreover, the multiple antigen standards (Solution B) are prepared as below:

| TM | STD1 | | STD2 | | STD3 | | STD4 | | STD5 | | STD6 | |
|---|---|---|---|---|---|---|---|---|---|---|---|---|
| SCCA | 0 | ng/ml | 2 | ng/ml | 20 | ng/ml | 200 | ng/ml | 1000 | ng/ml | 2000 | ng/ml |
| CA125 | 0 | U/ml | 40 | U/ml | 200 | U/ml | 600 | U/ml | 1200 | U/ml | 2400 | U/ml |
| CA15-3 | 0 | U/ml | 1 | U/ml | 10 | U/ml | 100 | U/ml | 400 | U/ml | 800 | U/ml |
| CEA | 0 | ng/ml | 2 | ng/ml | 20 | ng/ml | 200 | ng/ml | 1200 | ng/ml | 2400 | ng/ml |
| β-HCG | 0 | ng/ml | 2 | ng/ml | 20 | ng/ml | 200 | ng/ml | 1000 | ng/ml | 2000 | ng/ml |

Prepare Solution B according to the above table with PBS, pH7.4.
Moreover again, the preparations of Solution A, C and H are similar to steps in EXAMPLES 1&2.

Detect tumor markers in digestive system of patient sample by prepared reagents. The operations refer to EXAMPLE 2 and the detection results are as the chart below:

| | | | | | | HAMA |
|---|---|---|---|---|---|---|
| | SCCA | CA125 | CA15-3 | CEA | β-HCG | Indication |
| Std1 | 85 | 78 | 95 | 75 | 76 | 84 |
| Std2 | 198 | 155 | 354 | 214 | 225 | 94 |
| Std3 | 534 | 457 | 945 | 546 | 635 | 105 |
| Std4 | 1245 | 1145 | 2785 | 1345 | 2140 | 125 |
| Std5 | 3489 | 2875 | 5345 | 3575 | 4575 | 114 |
| Std6 | 7985 | 5978 | 9475 | 7540 | 8745 | 135 |
| Sample 1 | 3245 | 1257 | 4575 | 2475 | 1350 | 125 |
| Sample 2 | 104 | 785 | 157 | 650 | 900 | 134 |
| Sample 3 | 752 | 74 | 978 | 75 | 1240 | 116 |
| Sample 4 | 92 | 68 | 125 | 87 | 70 | 105 |
| Sample 5 | 85 | 95 | 6475 | 3215 | 96 | 95 |
| Sample 6 | 24573 | 450 | 27851 | 23565 | 27452 | 26485 |
| Sample 7 | 30215 | 27955 | 28450 | 23451 | 29785 | 27850 |
| Sample 8 | 29451 | 26785 | 26450 | 27459 | 23875 | 28950 |
| Sample 9 | 16470 | 10054 | 19075 | 15742 | 18642 | 147820 |
| Sample 10 | 21453 | 22078 | 25409 | 20985 | 22456 | 21765 |

Determination of the results: Samples 1-5 are without HAMA interference and Samples 6-10 are of HAMA interference.

### EXAMPLE 4

Indication of HD-HOOK Effect and Detection of AFP in Human Serum

The first and second antibodies specific to AFP are supplied by Shanghai Secondary Military Medical University, Antigen AFP is also supplied by Biodesign Corp. and Microbeads are supplied by Luminex Corp.

1. Preparation
1.1 Remove the primary amines and other small proteins (through dialysis or gel filtration) in the first antibody and antigen AFP solutions and measure their concentrations.
1.2 Precisely weight about 5 mg of N-hydroxysulfosuccinimide (NHS) to an polypropylene tube for later use (against moisture).
1.3 Precisely weight about 5 mg of N-ethyl-N′(3-dimethylainopropyl)-carbodiimide (EDC) to anpolypropylene tube for later use (against moisture).
2 The activation of the micro beads
2.1 Resuspend the stock microsphere suspension of 33# and 51# set by vortex for approximately 20 seconds. Transfer 200 μl of the stock microsphere suspension (about 2.5×10ˆ6 beads) respectively to two polypropylene tubes.
2.2 Pellet the microspheres by micro centrifugation at 15000 rpm for 2 mins (set to 3 mins), remove the supernatant.
2.3 Resuspend the pelleted microspheres in 100 μl dH₂O by vortex approximately 20 seconds, pellet the microspheres by micro centrifugation at 15000 rpm for 2 mins, and remove the supernatant.
2.4 Repeat step 2.3.
2.5 Resuspend the pelleted microspheres in 80 μl 0.1 mol/LPBS, pH6.2 by vortex approximately 20 seconds.
2.6-2.16 Conduct the same as in EXAMPLE 1.
3. Coupling, Blocking and Storage.
3.1 Add 20 μg of the prepared first antibody specific to AFP to the activated 33# set beads. Add 20 μg of the prepared Antigen AFP to the activated 51# set beads. Mix respectively by vortex for approximately 20 seconds.
3.2 Incubate at 37° C. for 2 hours against light with gentle mixing by vortex at 15 minute intervals.
3.3 Resuspend the pelleted microspheres in 1000 μl PBS-TBN by vortex approximately 20 seconds (PBS-TBN containing 10 mM PBS pH7.4, 02% of TWEEN20, 1 mg/ml Bovine Serum Albumin and 0.05% Sodium Azide).
3.4 Resuspend the pelleted microspheres in 500 μl PBS-TBN by vortex approximately 20 seconds and micro centrifugation at 15000 rpm for 2 mins, remove the supernatant.
3.5 Resuspend the pelleted microspheres in 500 μl PBS-TBN by vortex approximately 20 seconds.
3.6 Measure the beads concentrations by microscopic examination and cell count, the results are as hereafter: 33# is 1×10⁶/ml; 51# is 1×10⁶/ml.
3.7 Store at 2-8° C. against light
4. Biotinylation of Second Antibodies

4.1 Preparation of second antibodies

| | |
|---|---|
| Containing small molecules as | Dialyze against 1xPBS pH 7.4 |
| sodium azide, amines like glycine; | |
| Containing large molecules as | Purify by protein A column |
| bovine serum albumin | or other columns |
| Concentration calibrating | Measure OD280 (1OD280 equivalent |
| | to 0.7 mg/ml McAb) by |
| | spectrophotometer, and bring the |
| | concentration up to 2 mg/ml by |
| | 1xPBS, pH 7.4 (when concentrate, |
| | use desalting column from Pall Inc.). |

4.2 Biotinylating reaction
   Add 25 μl 1 mg/ml biotin DMSO solution into 10 μl the above second antibodies specific to AFP solution, mix gently and react in refrigerator at 4° C. for 2 hours against light. Dialysis overnight for later use.
5. Preparation of AFP antigen Standards (Solution B)
   Dissolve AFP antigen in 1×PBS, pH7.4 solution to prepare standards with concentrations of 0 ng/ml, 5 ng/ml, 20 ng/ml, 200 ng/ml, 500 ng/ml.
6. Preparation of Solution A
   Put 33# bead coupled with the first antibody here above in 1 ml PBS, pH7.4 solution with concentration of 1×10⁴/ml.
7. Preparation of PE Conjugated Second Antibody (Solution C)
   Mix the Biotinylated second antibody specific to AFP in PBS, pH7.4 solution with a concentration of 5 μg/ml, add Streptavidin linked PE in it with a total concentration of 60 μg/ml, and bring the total volume up to 5 ml, store at 4° C. against light for later use.
8. Preparation of Solution H
   Put 51# bead coupled with Antigen AFP here above in 1 ml PBS, pH7.4 solution with concentration of 1×10⁴/ml.
9. Indication of HD-HOOK effect and detection of AFP in human serum
9.1 Collect 5 samples with AFP concentration above 20000 ng/ml and another 5 samples with AFP concentration less than 1000 ng/ml, each of 2 ml/vial, centrifuge at 500 rpm for 5 mins and collect supernatant for later use.
9.2 Add separately Solution B, 5 μl; 25 μl Solution A, 25 μl Solution C to each well; and add 5 μl/well of 10 human serum samples, 25 μl/well Solution A, and 25 μl/well Solution C into other 10 wells. Mix and react in incubator at 37° C. for 10 mins.
9.3 After incubation, mix on vortex and read the plate on Lumine 100 analyzer.

9.4 Detection results refer to the chart below:

| | | Detection Results | |
|---|---|---|---|
| | Samples or known | Detection | 51# HD-HOOK |
| | concentrations | microsphere | Indication microsphere |
| Standards | 0 ng/ml | 56.7 | 11205.5 |
| | 5 ng/ml | 250.8 | 11304 |
| | 20 ng/ml | 768.8 | 11098 |
| | 200 ng/ml | 4829.5 | 11196 |
| | 500 ng/ml | 7439.5 | 11245 |
| Samples | Sample 1 | 7945 | 10020 |
| | Sample 2 | 5678.5 | 154 |
| | Sample 3 | 6745.5 | 230 |
| | Sample 4 | 3725 | 145 |
| | Sample 5 | 9875 | 285 |
| | Sample 6 | 115.0 | 12456 |
| | Sample 7 | 3478.0 | 12378 |
| | Sample 8 | 257.0 | 13795 |
| | Sample 9 | 6473.0 | 11245 |
| | Sample 10 | 578.5 | 11659.5 |

9.5 Determination of the results:
   The average MIF of 51# beads (HOOK Indication Bead) with different concentrations of standards is 11209.7, with 2SD of 151.0, making the normal MIF of Indication Bead about 11360.7; The average MIF of the Indication Bead with Samples 1-5 are all less than 90% of its normal MIF(10224.63), thus Samples 1-5 are determined to have HD-HOOK effect. Sample 1 is of slight HD-HOOK effect which results in a little low MIF, while Sample 2-4 are of remarkable HD-HOOK effect with much lower MFI (less than 10% of the normal MIF of Indication Bead) that result in false negative of detection results. Meanwhile, the MIF of HOOK Indication Bead with Samples 6-10 are of normal, they are samples without HD HOOK effect (the detection results is reliable without false negative caused by HD-HOOK effect).

### EXAMPLE 5

Simultaneously measurement of 5 kinds of tumor markers (With HD-HOOK Indication)

Generally according to the steps in EXAMPLES 1&4, detecting simultaneously 5 kinds of tumor markers with HD-HOOK interference indication. Different target antigens SCCA, CA125, CA15-3,CEA, β-HCG are used. Each uses one detection beads and HAMA indication microbeads. 5 detection microbeads and 5 indication microbeads have different coding fluorescence Moreover, the multiple antigen standards (Solution B) are prepared as below:

| TM | STD1 | | STD2 | | STD3 | | STD4 | | STD5 | | STD6 | |
|---|---|---|---|---|---|---|---|---|---|---|---|---|
| SCCA | 0 | ng/ml | 2 | ng/ml | 20 | ng/ml | 200 | ng/ml | 1000 | ng/ml | 2000 | ng/ml |
| CA125 | 0 | U/ml | 40 | U/ml | 200 | U/ml | 600 | U/ml | 1200 | U/ml | 2400 | U/ml |
| CA15-3 | 0 | U/ml | 1 | U/ml | 10 | U/ml | 100 | U/ml | 400 | U/ml | 800 | U/ml |
| CEA | 0 | ng/ml | 2 | ng/ml | 20 | ng/ml | 200 | ng/ml | 1200 | ng/ml | 2400 | ng/ml |
| β-HCG | 0 | ng/ml | 2 | ng/ml | 20 | ng/ml | 200 | ng/ml | 1000 | ng/ml | 2000 | ng/ml |

Prepare Solution B according to the above table with PBS, pH7.4.
Moreover again, the preparations of Solution A, C and H are similar to steps in EXAMPLE 1&2.

8.3 Detect tumor markers in digestive system of patient sample by prepared reagents. The operations refer to EXAMPLE 2 and the detection results are as the chart below:

| 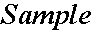 | 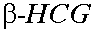 | 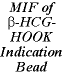 | 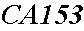 | 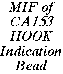 | 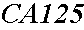 | 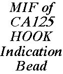 | 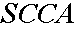 | 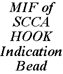 | 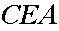 | 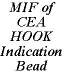 |
|---|---|---|---|---|---|---|---|---|---|---|
| 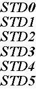 | 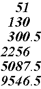 | 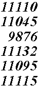 | 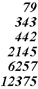 | 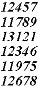 | 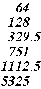 | 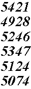 | 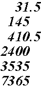 | 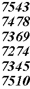 | 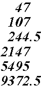 | 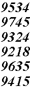 |
| normal MFI | 11896.0 | | 13356.33 | | 5537.13 | | 7631.7 | | 9872.82 | |
| of HOOK | | | | | | | | | | |
| Indication | | | | | | | | | | |
| Beads | | | | | | | | | | |
| Sample 1 | 81 | 12431 | 598 | 12475 | 125 | 5142 | 97 | 7542 | 411 | 9547 |
| Sample 2 | 5748 | 11456 | 35 | 13456 | 116.5 | 5147 | 37 | 7568 | 1419 | 9684 |
| Sample 3 | 68.5 | 11345 | 1271 | 12695 | 113.5 | 5263 | 4570 | 7648 | 150 | 9387 |
| Sample 4 | 66 | 12785 | 3915 | 15437 | 61 | 5347 | 64 | 7489 | 955 | 9574 |
| Sample 5 | 47.5 | 13456 | 102 | 14289 | 2154 | 5289 | 59 | 7398 | 1784 | 9471 |
| Sample 6 | 7421 | 11873 | 10471 | 15432 | 164.5 | 5478 | 120 | 7345 | 7459 | 7216 |
| Sample 7 | 124 | 13452 | 98 | 13487 | 4712 | 3621 | 5741 | 5487 | 421 | 9350 |
| Sample 8 | 5321 | 5483 | 7962 | 6425 | 458 | 1425 | 75 | 7456 | 58 | 9415 |
| Sample 9 | 75 | 14782 | 59 | 13756 | 982 | 1578 | 645 | 1459 | 6579 | 5215 |
| Sample 10 | 8035 | 7425 | 245 | 1457 | 3545 | 5374 | 3785 | 2478 | 1463 | 987 |

Determination of the results:
The average MIF with bold italic data in the chart above are of standards, others are of samples; the data with underline in the chart above are of samples with HD-HOOK effect.

### EXAMPLE 6

Simultaneously measurement of 5 kinds of tumor markers (With HD-HOOK Indication and HAMA Interference Indication)

Generally follow the steps in EXAMPLE 3&5 to conduct the measurement of 5 kinds of tumor markers in 20 samples of EXAMPLE 3&5, simultaneously with 33# set detection beads, 46# set HAMA Interference Indication beads, and 51# set beads for HD-HOOK Indication.

The results indicate that there is no intra-interference with every measure beads and it's reliable to detect samples with HAMA Interference or HD-HOOK.

### EXAMPLE 7

Assay Kit

Mix 4×10⁵ of hereafter 10 different kinds of beads as anti₁β-HCG-Beads, anti₁PEree-PSA-Beads, anti₁total-PSA-Beads, anti₁NSE-Beads, anti₁CA15-3-Beads, anti₁CA19-9-Beads, anti₁CA125-Beads, anti₁CA242-Beads, anti₁APEP-Beads, anti₁CEA-Beads from Example 1 respectively in a container of PBS, pH7.4 solution and bring the total volume up to 5 ml to prepare an assay kit for multiple tumor makers.

Moreover, put both 33# set of measure bead and 46# set of HAMA Indication bead which are prepared in EXAMPLE 1&2 separately in the container above to prepare an assay kit with HAMA Indication.

Moreover again, put 51# set of HD-HOOK Indication bead which is prepared in EXAMPLE 4 in another container thus to prepare an assay kit with HD-HOOK Indication simultaneously.

### EXAMPLE 8

Simultaneously measurement of 6 kinds of tumor markers

Steps 1-4 follow that in EXAMPLE 1.

5. Preparation of multiple antigen Standards and Controls (Solution B)

| TM | STD0 | | STD1 | | STD2 | | STD3 | | STD4 | | STD5 | | Ctr1 | | Ctr2 | |
|---|---|---|---|---|---|---|---|---|---|---|---|---|---|---|---|---|
| CA19-9 | 0 | U/ml | 5 | U/ml | 40 | U/ml | 200 | U/ml | 1000 | U/ml | 2000 | U/ml | 40 | U/ml | 200 | U/ml |
| CA125 | 0 | U/ml | 40 | U/ml | 200 | U/ml | 600 | U/ml | 1200 | U/ml | 2400 | U/ml | 200 | U/ml | 600 | U/ml |
| CA50 | 0 | U/ml | 5 | U/ml | 40 | U/ml | 200 | U/ml | 1000 | U/ml | 2000 | U/ml | 40 | U/ml | 200 | U/ml |
| AFP | 0 | ng/ml | 5 | ng/ml | 20 | ng/ml | 200 | ng/ml | 500 | ng/ml | 500 | ng/ml | 20 | ng/ml | 200 | ng/ml |
| CEA | 0 | ng/ml | 2 | ng/ml | 20 | ng/ml | 200 | ng/ml | 1200 | ng/ml | 2400 | ng/ml | 20 | ng/ml | 200 | ng/ml |
| CA72-4 | 0 | ng/ml | 2 | ng/ml | 20 | ng/ml | 200 | ng/ml | 1000 | ng/ml | 2000 | ng/ml | 20 | ng/ml | 200 | ng/ml |

Dissolve the above antigens in PBS, pH7.4 solution.
6. Preparation of Solution A
   Mix 4×10⁵ of hereafter 10 different kinds of beads as anti₁CA199-Beads, anti₁CA125-Beads, anti₁CA50-Beads, anti₁CA72-4-Beads, anti₁AFP-Beads, anti₁CEA-Beads respectively in PBS, pH7.4 solution and bring the total volume up to 5 ml, store at 4° C. against light for later use.
7. Preparation of the Mixture of PE Conjugated Second Antibodies (Solution C)
   Mix the Biotinylated second antibodies specific to CA19-9, CA125, CA50, CA72-4, AFP and CEA respectively in 1×PBS, pH7.4 solution each with a concentration of 5 μg/ml, add PE with a total concentration of 60 μg/ml, and bring the total volume up to 5 ml, store at 4° C. against light for later use.
8. Detection for tumor markers in digestive system of patient sample
8.1 Collect 6 blood samples from patient of 2 ml/vial, centrifuge at 5000 rpm for 5 mins and collect supernatant for later use.
8.2 Add separately Solution A, 50 μl; Standards (STD0, STD1, STD2, STD3, STD4, STD5), Controls (Ctr1, Ctr2) and Serum Samples (NO.1-6), each 50 μl/well; and Solution C, 50 μl to each well in a 96 well plate. Mix gently on vortex and incubate on a plate shaker at 37° C. for 40 mins.
8.4 After incubation, mix on vortex and read the plate on Lumine100 analyzer.

8.4 Detection results refer to the chart below:

| Location | 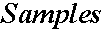 | 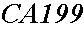 | 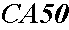 | 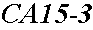 | 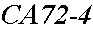 | 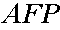 | 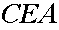 | Total Events |
|---|---|---|---|---|---|---|---|---|
| 1 2 3 4 5 | 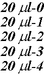 | 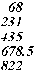 | 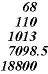 | 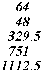 | 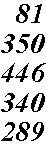 | 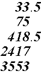 | 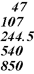 | 1286 1243 1282 1268 1359 |
| 6 | Sample 1 | 56 | 146 | 35 | 43 | 279 | 75 | 1253 |
| 7 | Sample 2 | 65.5 | 126 | 92 | 50 | 778 | 54.5 | 1666 |
| 8 | Sample 3 | 28.5 | 69 | 30 | 60.5 | 102 | 67 | 1346 |
| 9 | Sample 4 | 39 | 73 | 60 | 71.5 | 87 | 22.5 | 1312 |
| 10 | Sample 5 | 67 | 106 | 62 | 99 | 169 | 42.5 | 1341 |
| 11 | Sample 6 | 51.5 | 157 | 100 | 222 | 393 | 19 | 1392 |

NOTE: The MIF with bold italic data in the chart above are of standards, others are of samples. The results indicate that it is feasible of simultaneous and quantitative detection of multiple tumor markers by the method of present invention, for example, there is large amount of tumor marker APFP in Sample 7 of the chart above and this result can assist clinical diagnosis.

### EXAMPLE 9

Simultaneously measurement of 5 kinds of tumor markers

Steps 1-4 follow that in EXAMPLE 1.

5. Preparation of multiple antigen Standards and Controls (Solution B)

| TM | STD0 | | STD1 | | STD2 | | STD3 | | STD4 | | STD5 | | Ctr1 | | Ctr2 | |
|---|---|---|---|---|---|---|---|---|---|---|---|---|---|---|---|---|
| SCCA | 0 | ng/ml | 2 | ng/ml | 20 | ng/ml | 200 | ng/ml | 1000 | ng/ml | 2000 | ng/ml | 20 | ng/ml | 200 | ng/ml |
| CA125 | 0 | U/ml | 40 | U/ml | 200 | U/ml | 600 | U/ml | 1200 | U/ml | 2400 | U/ml | 200 | U/ml | 600 | U/ml |
| CA15-3 | 0 | U/ml | 1 | U/ml | 10 | U/ml | 100 | U/ml | 400 | U/ml | 800 | U/ml | 10 | U/ml | 100 | U/ml |
| CEA | 0 | ng/ml | 2 | ng/ml | 20 | ng/ml | 200 | ng/ml | 1200 | ng/ml | 2400 | ng/ml | 20 | ng/ml | 200 | ng/ml |
| β-HCG | 0 | ng/ml | 2 | ng/ml | 20 | ng/ml | 200 | ng/ml | 1000 | ng/ml | 2000 | ng/ml | 20 | ng/ml | 200 | ng/ml |

Dissolve the above antigens in PBS, pH7.4 solution.
6. Preparation of Solution A
   Mix 4×10⁵ of hereafter 10 different kinds of beads as anti₁SCCA-Beads, anti₁CA15-3-Beads, anti₁CA125-Beads, anti₁CEA-Beads anti₁β-HCG-Beads respectively in PBS, pH7.4 solution and bring the total volume up to 5 ml, store at 4° C. against light for later use.
7. Preparation of the Mixture of PE Conjugated Second Antibodies (Solution C)
   Mix the Biotinylated second antibodies specific to SCCA, CA15-3, CA125, CEA and β-HCG respectively in 1×PBS, pH7.4 solution each with a concentration of 5 μg/ml, add PE in it with a total concentration of 60 μg/ml, and bring the total volume up to 5 ml, store at 4° C. against light for later use.
8. Detection for tumor markers in digestive system of patient sample
8.1 Collect 6 blood samples from patient of 2 ml/vial, centrifuge at 5000 rpm for 5 mins and collect supernatant for later use.

8.2 Add separately Solution A, 50 μl; Standards (STD0, STD1, STD2, STD3, STD4, STD5), Controls (Ctr1, Ctr2) and Serum Samples (NO.1-6), each 20 μl; and Solution C, 50 μl to each well in a 96 well plate. Mix gently on vortex and incubate on a plate shaker at 37° C. for 40 mins 8.3 After incubation, mix on vortex and read the plate on Lumine 100 analyzer. 8.4 Detection results refer to the chart below:

| Location | Samples | 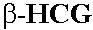 | 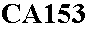 | 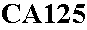 | 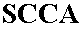 | 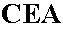 | Total Events |
|---|---|---|---|---|---|---|---|
| 1 2 3 4 5 | 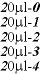 | 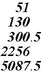 | 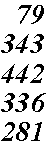 | 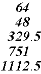 | 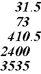 | 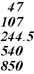 | 1286 1243 1282 1268 1359 |
| 6 | 1 | 81 | 598 | 125 | 97 | 411 | 1150 |
| 7 | 2 | 51 | 35 | 116.5 | 37 | 1419 | 1226 |
| 8 | 3 | 68.5 | 1271 | 113.5 | 109 | 150 | 1265 |
| 9 | 4 | 66 | 49 | 61 | 64 | 955 | 1451 |
| 10 | 5 | 47.5 | 102 | 66 | 59 | 1784 | 1235 |
| 11 | 6 | 38.5 | 133.5 | 164.5 | 120 | 198 | 1429 |

NOTE: The MIF with bold italic data in the chart above are of standards, others are of samples.

The results indicate that it is feasible of simultaneous and quantitative detection of multiple tumor markers by the method of present invention, for example, there is large amount of tumor marker CA15-3 in Sample 8 of the chart above and this result can assist clinical diagnosis.

All the documents cited herein are incorporated into the invention as reference, as if each of them is individually incorporated. Further, it would be appreciated that, in the above teaching of invention, the skilled in the art could make certain changes or modifications to the invention, and these equivalents would still be within the scope of the invention defined by the appended claims of the application.

What is claimed is:

1. A method for simultaneously detecting multiple antigens which are tumor markers, wherein the method comprises the following steps:
(a) mixing an analyte sample with a first antibody solution and a second antibody solution to form reaction mixture (a),
wherein mixing the sample with the first and the second antibody solutions can be conducted in order or simultaneously;
the first antibody solution contains 2 to 50 kinds of different antibodies, each first antibody is an antibody specific to tumor markers respectively and is coupled with distinct microsphere to form the composite as shown in Formula (I),
Anti₁X-bead (I)
wherein X represents a tumor marker, “Anti₁X” represents the first antibody that specific to the tumor marker X, and “bead” represents a microsphere and “-” represents a covalent bond;
the second antibody solution contains 2 to 50 kinds of different antibodies, each second antibody is specific to a tumor marker and corresponds to a first antibody in the first antibody solution, and each pair of the first antibody and second antibody can simultaneously bond to the corresponding tumor marker; and the mole ratio of first antibodies and the second antibodies is from 1:0.1 to 1:2;
thereby forming a quaternary composite of “Second antibody-Tumor Marker-First antibody-bead” in the reaction mixture (a);
(b) detecting the detectable signals from the bead of the quaternary composite to determine the presence of each tumor marker in the sample.

2. The method of claim 1 which further comprises the step (c): comparing the detectable signal measured with the control or the calibration curve to determine the presence and/or the amount of the tumor markers in the analyte sample.

3. The method of claim 1 wherein the detectable signal is fluorescence.

4. The method of claim 1 wherein the first antibody solution and the second antibody solution respectively contain the grouped first antibodies and the grouped second antibodies specific to the grouped tumor markers selected from the group consisting of:
(i) Alpha-Fetoprotein (AFP), Carcinoembryonic antigen (CEA), Carcinoma antigen 125 (CA125), Carbohydrate antigen 199 (CA199), total Human prostate antigen (PSA), free Human prostate antigen (f-PSA), Neuron specific enolase (NSE), Carbohydrate antigen 242 (CA242), Cancer antigen 153 (CA153), beta human Chorionic Gonadotropin (β-HCG);
(ii) Alpha-Fetoprotein (AFP), Carcinoembryonic antigen (CEA), Carcinoma antigen 125 (CA125), Carbohydrate antigen 199 (CA199), Carbohydrate antigen 72-4 (CA72-4), Carbohydrate antigen 50 (CA50);
(iii) Carcinoembryonic antigen (CEA), Carcinoma antigen 125 (CA125), Neuron specific enolase (NSE), beta human Chorionic Gonadotropin (β-HCG), Carbohydrate antigen 50 (CA50), Squamous cell carcinoma antigen (SCCA), Cytokeratin 19 (CYFRA21-1);
(iv) Carcinoembryonic antigen (CEA), Carcinoma antigen 125 (CA125), Cancer antigen 153 (CA153), beta human Chorionic Gonadotropin (β-HCG), Squamous cell carcinoma antigen (SCCA); and
(v) total Human prostate antigen (PSA), free Human prostate antigen (f-PSA).

5. The method of claim 1 wherein the concentration of each first antibody is from 1 to 100 ug/ml in the first antibody solution; and the concentration of each second antibody is from 0.1 to 200 ug/ml in the second antibody solution.

6. The method of claim 1 wherein the step (a) further includes the adding HD-HOOK indication bead into the reaction mixture (a), wherein the HD-HOOK indication bead comprises a binary composite of “target antigen-beads” as shown in Formula (II),
X-bead′ (II)
wherein the “bead′” represents a microsphere different from the above “bead” in Formula (I), and “-” represents the linkage between X and “bead′”,
thereby forming a ternary composite of “Second antibody-Antigen-Indication bead′” under the existence of the second antibody having detectable signals; and
step (b) comprises detecting the signal on the bead of the quaternary composite and comparing the signals with the standard value or the calibration curve to determine the presence and/or the amount of the target antigen in the analyte sample; and
detecting the signal on the bead of the ternary composite and comparing the signals of HD-HOOK indication bead with the normal value with no HOOK effect, wherein if the signal of the HOOK bead is smaller than the normal signal, it is indicated that the measurement result is incredible; and if the signal of the HOOK bead is larger than or equivalent to the normal value, it is indicated that the concentration of the target antigen is in the measurable range.

7. The method of claim 1 wherein the step (a) further includes adding Heterophile Antibody Interference indication bead to the reaction mixture (a), wherein the Heterophile Antibody Interference indication bead is a binary composite of “Heterophile Antibodies Interference Catcher-bead” as shown in Formula (III),
Z-bead″ (III)
wherein “Z” represents the Heterophile Antibodies Interference Catcher, “bead″” represents a microsphere different from the above “bead” and “bead′”, and “-” represents the linkage between “Z” and “bead″”,
thereby forming a quaternary composite of “Second antibody—Heterophile Antibodies—Heterophile Antibodies Catcher-bead″” when the Heterophile Antibodies exist in the sample; and
step (c) comprises detecting the detectable signal on the bead of the quaternary composite and comparing the signals with the standard value or the calibration curve, thereby determining the presence and/or the amount of the target antigen in the analyte sample; and
detecting the detectable signal on the bead of the quaternary composite of “Second antibody—Heterophile Antibodies—Heterophile Antibodies Catcher-bead″” and comparing the signals of Heterophile Antibodies Catcher-bead″ with the normal value (without Heterophile Antibodies interference effect), wherein if the signal of the Heterophile Antibodies Catcher-bead″ is >1.5 times of the normal value, it is indicated that the measurement result is incredible; and if the signal of the Heterophile Antibodies Catcher-bead″ is [symbol] 1.5 times of the normal value, it is indicated that there is no interference of the Heterophile Antibodies in the sample.

8. A kit for detecting multiple tumor markers which comprises the following components:
(a′) a first vessel which contains a first antibody solution, wherein the first antibody solution contains 2 to 50 kinds of different first antibodies, and each first antibody is specific to a tumor marker respectively and is coupled onto distinct set of microsphere to become a binary composite of first Antibody-bead as shown in Formula (I),
Anti₁X-bead (I)
wherein X represents a tumor marker, “Anti₁X” represents the first antibody specific to tumor marker X, “bead” represents a microsphere and “-” represents a covalent bond.
(b′) a second vessel which contains a second antibody solution, wherein the second antibody solution contains 2 to 50 kinds of different antibodies having different detectable signals, each second antibody is specific to a tumor marker and corresponds to a first antibody in the first antibody solution, each pair of the first antibody and second antibody can simultaneously bond to the corresponding tumor marker, and the mole ratio of first antibodies and the second antibodies is from 1:0.1 to 1:2.

9. The kit of claim 8 wherein the kit further includes the indication beads selected from the group consisting of:
(c′) a HD-HOOK indication bead, wherein the HD-HOOK indication bead comprises a binary composite of “target antigen-beads” as shown in Formula (II),
X-bead′ (II)
wherein the “bead′” represents a microsphere different from the above “bead” in Formula (I), and “-” represents the linkage between X and “bead′”;
(d′) a Heterophile Antibody Interference indication bead, wherein the Heterophile Antibody Interference indication bead is a binary composite of “Heterophile Antibodies Interference Catcher-bead” as shown in Formula (III),
Z-bead″ (III)
Wherein, “Z” represents the Heterophile Antibodies Interference Catcher selected from the group consisting of rheumatoid factors or antibodies from mouse, chick, rabbit, goat, house, or cow, “bead″” represents a nicrosphere different from the above “bead” and “bead′”, and “-” represents the linkage between “Z” and “bead″”.

10. A use of the kit as defined in claim 8 in an in vitro detection of the presence of tumor markers in a sample.
